(19) United States
(12) Patent Application Publication
(10) Pub. No.: US 2015/0065709 A1
(43) Pub. Date: Mar. 5, 2015

(54) Aminoquinazoline Derivative And Use Thereof In Preparing Anti-Malignant Tumor Medicament

(71) Applicant: METABOMICS, INC, Zhangjiagang, Jiangsu (CN)

(72) Inventors: Jianming Yin, Zhangjiagang (CN); Junhua Tao, Zhangjiagang (CN)

(21) Appl. No.: 14/385,734

(22) PCT Filed: Mar. 13, 2013
(86) PCT No.: PCT/CN2013/072528

§ 371 Date: Sep. 16, 2014

(30) Foreign Application Priority Data
Mar. 16, 2012 (CN) 201210060348.7

(51) Int. Cl. C07D 239/94; C07D 405/12; C07D 401/12; C07D 405/14

(52) U.S. Cl. CPC C07D 239/94; C07D 405/14; C07D 405/12; C07D 401/12
USPC 544/284; 544/293

(57) ABSTRACT

The invention discloses a new amino-quinazoline derivative and its use in preparing drugs for preventing and/or treating malignancies. The amino-quinazoline derivative of the invention is an ideal, high effective, dual and irreversible EGFR and HER2 kinase inhibitor, and can treat or prevent various malignancy diseases, such as breast cancer, ovarian cancer, gastrointestinal cancer, oesophageal cancer, lung cancer, head and neck squamous cancer, pancreatic cancer, epidermis squamous cell cancer, prostatic cancer, neuroglioma and nasopharynx cancer.

### FIELD OF THE INVENTION

The present invention relates to an amino-quinazoline derivative and its use in manufacture of medicaments for use in the prevention and/or treatment of malignant tumors.

### DESCRIPTION OF THE RELATED ART

Epidermal growth factor receptor (EGFR, HER1/erbB1) family is one of the most representative molecules of the transmembrane receptor tyrosine kinases, and has extensive biological functions. Various ligands, such as epidermal growth factor and transforming growth factor-α, can be bound with extracellular portions of EGFR cells to transfer the mitosis signals into cells, to further adjust the cell cycle and normal cell differentiation and accelerate the repair of damage. Furthermore, EGFR also can activate downstream vascular epidermal growth factor receptors (VEGFRs) and accelerate the forming of capillary network of solid tumors. Therefore, EGFR plays a significant role in occurrence, development, differentiation, repair and transfer of tumour cells. Studies show that abnormal activation, amplification and over-expression of EGFR genes generally are existing in various tumors derived from epithelial cells, such as breast cancer, colorectal cancer, head and neck squamous cancer, and pancreatic cancer. There are a lot of studies using EGFR as a therapeutic target, and wherein the monoclonal antibody and tyrosine kinase inhibitors (TKIs) are most successful. TKIs act on the interior of EGFR cells and competitively bind with ATP, to inhibit activity and phosphorylation of kinases, and to enclose the binding site of EGFR tyrosine kinases and ATP, thereby achieving the specific inhibition of EGFR.

Human epidermal growth factor receptor-2 (HER2/neu, erbB-2) is a transmembrane receptor, and is over-expressed in many epidermal tumors such as breast cancer, ovarian cancer, prostatic cancer, non-small cell lung cancer and nasopharynx cancer, and the over-expression of HER2/neu gene is existing in approximate 25%-30% of primary breast cancers.

### SUMMARY OF THE INVENTION

A technical problem to be actually solved by the invention is to provide a new amino-quinazoline derivative, which is an ideal EGFR and HER2 kinase inhibitor, and can be used for effectively preventing or treating various malignant tumor diseases, such as breast cancer, ovarian cancer, gastrointestinal cancer, oesophageal cancer, lung cancer, head and neck squamous cancer, pancreatic cancer, epidermis squamous cancer, prostatic cancer, neuroglioma and nasopharynx cancer.

In order to solve the above technical problem, the following technical solutions are utilized:

A compound of the Formula (I), a pharmaceutically acceptable salt or hydrate thereof, a prodrug thereof, or a metabolite thereof produced in any type of metabolism,

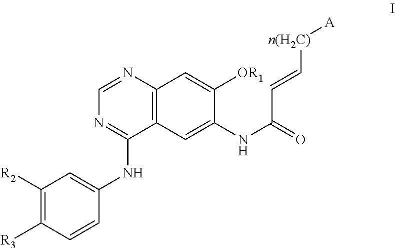

wherein:
R₁ is —CH₂F, —CHF₂, C₂-C₁₂ fluorinated hydrocarbyl, C₂-C₁₂ chlorinated hydrocarbyl; or
R₁ is —CₖH₂ₖ₋₁O, wherein k is an integer between 3 and 6;
R₂ is F, Cl, C₁-C₁₂ saturated alkyl or C₂-C₁₂ unsaturated alkenyl or alkynyl or alkenynyl;
R₃ is F, Cl, —OCᵢH₂ᵢC₅H₄N, —OCᵢH₂ᵢC₄H₃N₂ or —OCᵢH₂ᵢC₃H₂N₃, wherein i is an integer between 1 and 4;
n is 0, 1, 2 or 3;

A is a 5-membered or 6-membered saturated or unsaturated, substituted or unsubstituted heterocyclic structure, or a substituted or unsubstituted benzene ring; or A is —NR₅R₆, wherein R₅ and R₆ are independently selected from H and C₁-C₁₂ hydrocarbyl, and wherein in the compound of the Formula (I), the pharmaceutically acceptable salt or hydrate thereof, the prodrug thereof or the metabolite thereof produced in any type of metabolism, non-exchangeable hydrogen is not substituted, or partly or fully substituted by deuterium.

According to one aspect of the invention, in the Formula (I), R₁ is selected from the group consisting of —CHF₂, chlorinated ethyl and tetrahydrofuryl, and R₂, R₃, n and A are defined as above.

According to another aspect of the invention, in the Formula (I), R₂ is Cl, R₃ is F, and R₁, n and A are defined as above.

According to a further aspect of the invention, in the Formula (I), n is 0 or 1, and R₁, R₂, R₃ and A are defined as above.

According to a specific and preferable aspect of the invention, in the formula (I), A is selected from the group consisting of —N(CH₃)₂, —N(CH₃)CH₂CH₃, —N(CH₃)CH₂Ph, imidazolyl, pyridyl, unsubstituted or alkyl-substituted butyrolactam and valerolactam, and R₁, R₂ R₃ and n are defined as in claim 1.

According to a specific aspect of the invention, A is

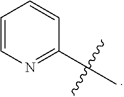 [SMILES: *c1ccccn1 ; "."]

According to the invention, the representative compounds are provided as follows:

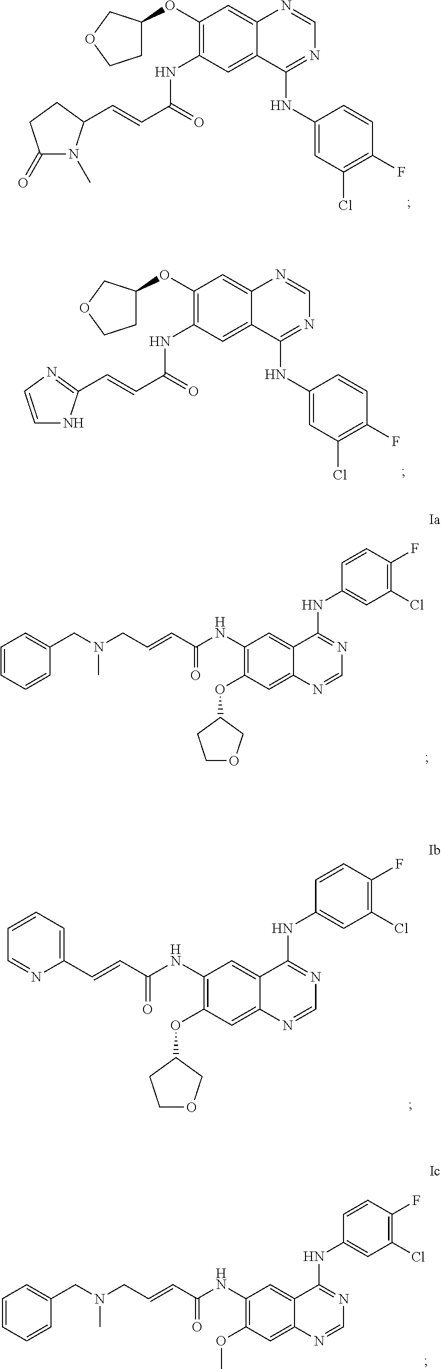

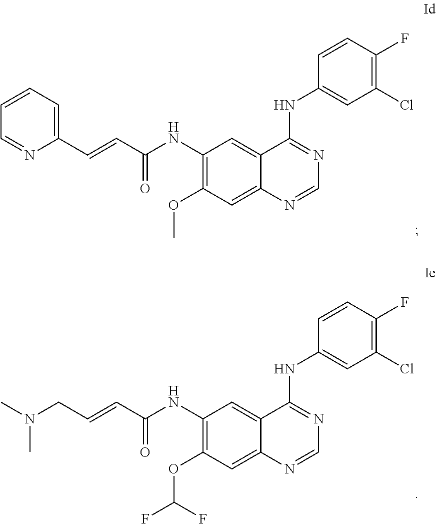 [SMILES: COc1cc2ncnc(Nc3ccc(F)c(Cl)c3)c2cc1NC(=O)/C=C/c1ccccn1 ; CN(C)C/C=C/C(=O)Nc1cc2c(Nc3ccc(F)c(Cl)c3)ncnc2cc1OC(F)F]

According to the invention, the compound includes not only a single certain compound, but also a mixture of compounds meeting the formula (1), and different isomers of a compound, such as a racemate, an enantiomer, and a diastereoisomer. The pharmaceutically acceptable salt includes (but not limited to) hydrochloride, phosphate, sulfate, acetate, maleate, mesylate, benzene sulfonate, benzoate, toluene sulfonate, succinate, fumarate, tartrate, gallate, citrate and the like. The “prodrug of the compound of the Formula (I)” refers to such a substance: after being applied in suitable methods, it can be converted into at least one compound of Formula (I) or salt thereof through metabolism or a chemical reaction within the body of a subject.

According to the invention, unless stated otherwise, the generic term “alkyl” refers to a straight-chain, branched-chain or cyclic hydrocarbyl, for example alkyls, including but not limited to, methyl, ethyl, n-propyl, isopropyl, isobutyl, tert-butyl, cyclopentyl, cyclohexyl or the like. Unless stated otherwise, the “hydrocarbyl” includes aliphatic hydrocarbyl and aromatic hydrocarbyl, and wherein the aliphatic hydrocarbyl may be in the form of a straight-chain, branched-chain or ring.

The chemical group —CₖH₂ₖ₋₁O covers all of the groups meeting this Formula, and is attached to other groups via hyphen “-”. A representative group of CₖH₂ₖ₋₁O is tetrahydrofuryl.

The compounds of the invention can be prepared by means of synthesis routes in usual methods well known in the field of chemistry, particularly, the compounds of the invention are synthesized according to the description contained herein. Reagents generally are commercially available or may be prepared using conventional methods well known for a person skilled in the art.

By adopting the above technical solutions, as compared with the prior art the invention has the following advantages:

The invention provides new amino-quinazoline derivatives, which are ideal highly effective, dual and irreversible tyrosine kinase inhibitors. By means of acting on the interior of EGFR cells and competitive binding with ATP, the amino-quinazoline derivatives of the invention can inhibit the activity and phosphorylation of kinases and enclose the binding site of EGFR tyrosine kinase with ATP, thereby achieving the specific inhibition of EGFR. Accordingly, the compounds of the invention can be used for preparing a drug for treatment or prevention various indications associated with EGFR and HER2 kinase functions, including but not limited to, various malignant tumor diseases, such as breast cancer, ovarian cancer, gastrointestinal cancer oesophageal cancer lung cancers head and neck squamous cancer pancreatic cancers epidermis squamous cancer, prostatic cancer, neuroglioma and nasopharynx cancer.

### BRIEF DESCRIPTION OF THE DRAWINGS

FIG. 1 is a contrast diagram showing the irreversible inhibitory effect of the compound of Formula Ie on activity of the EGFR kinases according to the invention;

FIG. 2 is a contrast diagram showing the inhibitory effect of the compound of Formula Ie on H1975 tumor growth in the nude mouse model according to the invention;

FIG. 3 is another contrast diagram showing the inhibitory effect of the compound of Formula Ie on H1975 tumor growth in the nude mouse model according to the invention;

FIG. 4 is a contrast diagram showing the inhibitory effect of the compound of Formula Ie of on A549 tumor growth in the nude mouse model according to the invention;

FIG. 5 is a contrast diagram showing the inhibitory effect of the compound of Formula Ie on HCC827 tumor growth in the nude mouse model according to the invention;

FIG. 6 is a contrast diagram showing the inhibitory effect of the compound of Formula Ie on A431 tumor growth in the nude mouse model according to the invention;

FIG. 7 is a contrast diagram showing the inhibitory effect of the compound of Formula Ie on N87 tumor growth in the nude mouse model according to the invention;

FIG. 8 is a contrast diagram showing the inhibitory effect of the compound of Formula Ie on MDA-MB-468 tumor growth in the nude mouse model according to the invention;

FIG. 9 is a contrast diagram showing the inhibitory effect of the compound of Formula Ie on CAL27 tumor growth in the nude mouse model according to the invention.

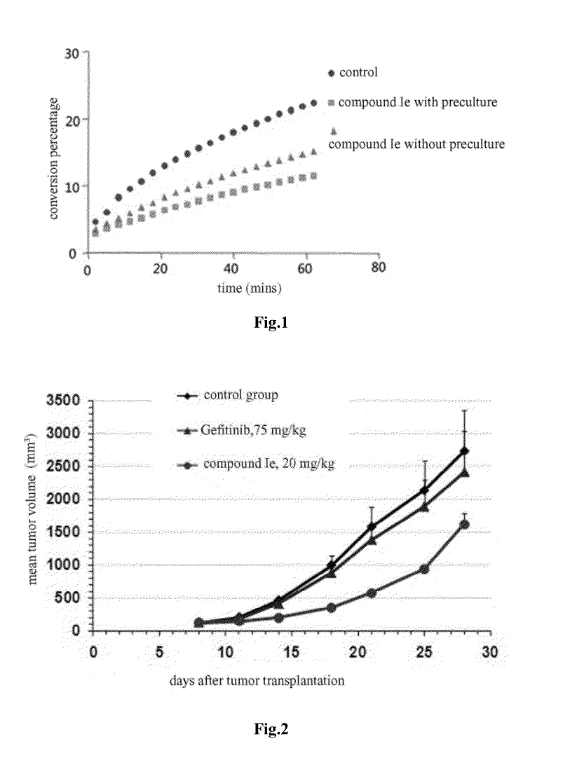

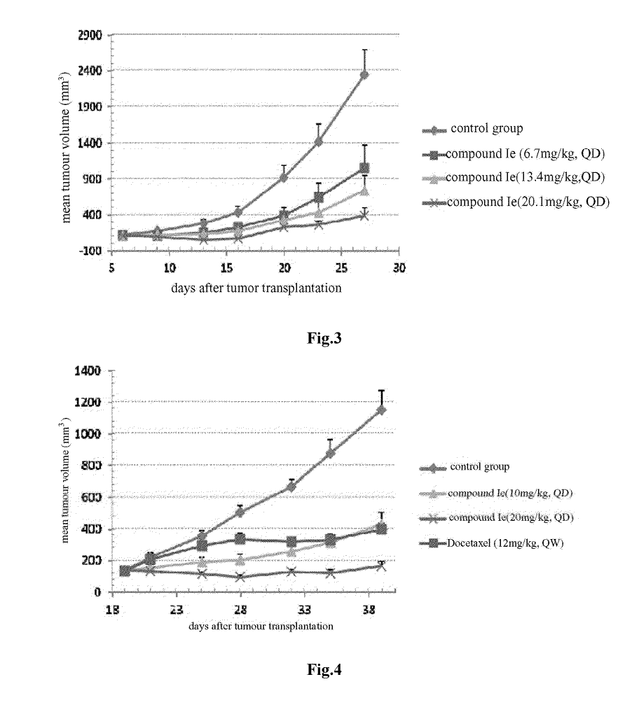

### DESCRIPTION OF THE PREFERRED EMBODIMENTS

The invention will now be illustrated in more detail in connection with specific embodiments hereinafter, however, it shall be appreciated that the present invention is not limited the following embodiments.

### Embodiment 1

The compound of Formula Ia has the following chemical formula:

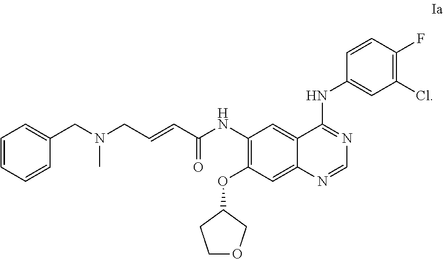

The compound of formula Ia can be obtained using the following synthesis route:

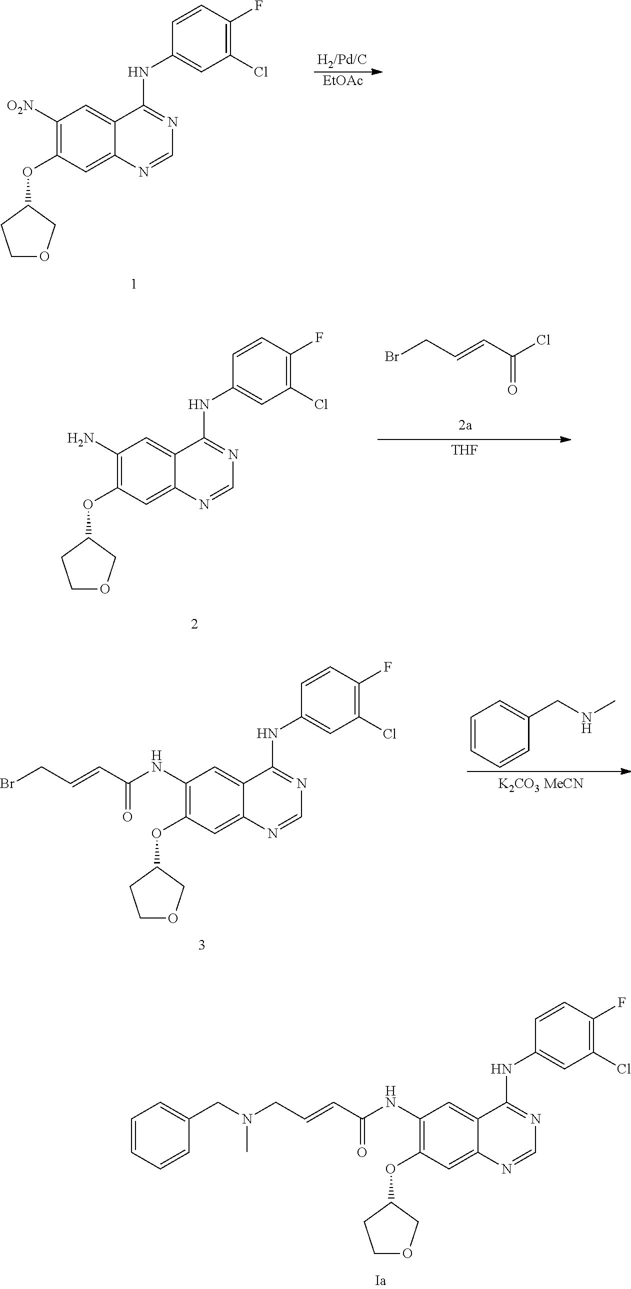

The preparation method of the compound of Formula Ia specifically comprises the steps of:

(1) preparation of intermediate 2: palladium on carbon (500 mg, 10 wt. %) was slowly added into the solution of the intermediate 1 (4 g, 9.9 mmol) in ethyl acetate (100 mL) at room temperature; the mixture was stirred in the presence of H₂ for 12 hours; subsequently the resulting solution was filtered, concentrated and dried under vacuum to obtain the intermediate 2 (3.7 g crude product, 100%)[m/s: [MH]⁺: 375.2]. The crude product of intermediate 2 was directly used in next step.
(2) preparation of intermediate 3: the solution of the intermediate 2a (0.61 g, 3.33 mmol) in tetrahydrofuran (10 mL) was slowly added to the intermediate 2 (0.5 g, 1.33 mmol) under stirring at 0° C.; the mixture was slowly heated to room temperature and further stirred for 3 hours; subsequently saturated sodium bicarbonate was added to stop the reaction and dilute the resulting mixture, and the solution was extracted with ethyl acetate; finally the organic phase was washed with saturated sodium chloride solution, dried with anhydrous sodium sulfate, filtered, concentrated, dried under vacuum and purified by silica gel chromatograph to obtain intermediate 3 (500 mg, 71.8%) [m/s: [MH]⁺: 522.7].
(3) preparation of compound Ia: the intermediate 3 (500 mg, 0.96 mmol) was slowly added to the solution of N-methylbenzylamine (350 mg, 2.9 mmol) and potassium carbonate (264 mg, 1.9 mmol) in acetonitrile (20 mL); the mixture was stirred for 12 hours and then diluted with water, extracted with ethyl acetate; the organic phase was washed with saturated sodium chloride solution, dried with anhydrous sodium sulfate, filtered, concentrated, dried under vacuum, and purified to get the title product (180 mg, 33.4%).

The obtained title product Ia was identified by H-nuclear magnetic resonance ¹H-NMR (400 MHz, MeOD) and mass spectrum analysis, the results are as follows:

Absorption peaks in ¹H-NMR spectrum: δ 8.49 (s, 1H), 8.16 (s, 1H), 7.72-7.71 (dd, 1H), 7.38-7.36 (dd, 1H), 7.19-7.12 (m, 5H), 6.93-6.83 (m, 2H), 6.72 (s, 1H), 6.32-6.28 (d, 1H), 5.35 (s, 1H), 4.89 (s, 1H), 3.95-3.69 (m, 6H), 3.06-3.05 (d, 2H), 2.21-2.18 (m, 1H), 2.09 (s, 3H), 2.04-1.99 (m, 1H).

m/s: [MH]⁺: 562.2. Through calculation, it is confirmed that the product has the molecular Formula of C₃₀H₂₉ClFN₅O₃, and the exact mass is 561.19.

### Embodiment 2

The compound of Formula Ib has the following chemical formula:

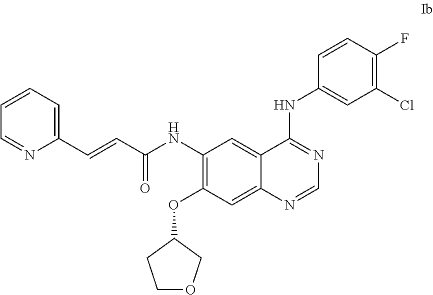

The compound of Formula Ib can be obtained by means of the substitution reaction of the intermediate 3 with the intermediate 3a, and the specific preparation process can be seen in embodiment 1.

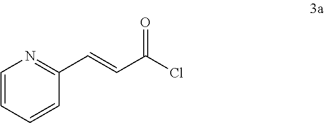 [SMILES: O=C(Cl)/C=C/c1ccccn1]

The obtained title product Ib was identified by ¹H-NMR (400 MHz, MeOD) and mass spectrum analysis, the results are as follows:

Absorption peaks in ¹H-NMR spectrum: δ 8.85 (s, 1H), 8.52-8.51 (dd, 1H), 8.376 (s, 1H), 7.93-7.91 (dd, 1H), 7.79-7.75 (t, 1H), 7.68-7.65 (d, 1H), 7.60-7.57 (dd, 2H), 7.31-7.27 (m, 2H), 7.15-7.11 (t, 1H), 7.06 (s, 1H), 5.17 (s, 1H), 4.11-4.08 (d, 1H), 4.02-3.92 (m, 2H), 3.85-3.79 (m, 1H), 2.36-2.33 (m, 1H), 2.20-2.17 (m, 1H).

Wherein m/s: [MH]⁺: 506.2. Through calculation, it is confirmed that the product has the molecular formula of C₂₆H₂₁ClFN₅O₃, and the exact mass is 505.13.

### Embodiment 3

The compound of Formula Ic has the following chemical formula:

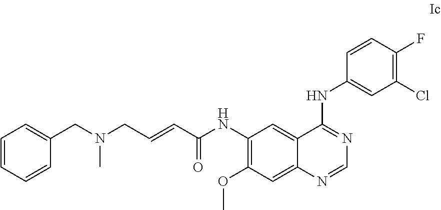 [SMILES: COc1cc2ncnc(Nc3ccc(F)c(Cl)c3)c2cc1NC(=O)/C=C/CN(C)Cc1ccccc1]

The compound of Formula Ic can be obtained using the following synthesis route:

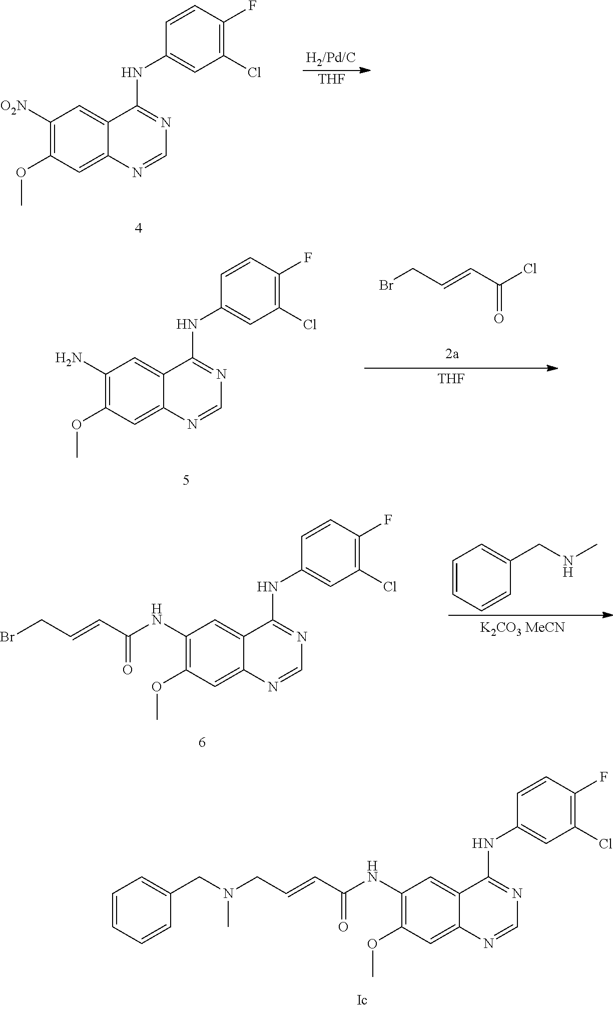

The preparation method of the compound of Formula Ic specifically comprises the steps of:

(1) preparation of intermediate 5: palladium on carbon (500 mg, 10 wt. %) was slowly added into the solution of the intermediate 4 (3 g, 8.6 mmol) in tetrahydrofuran (30 mL) at room temperature; the mixture was stirred in the presence of H₂ for 12 hours; subsequently the resulting solution was filtered, concentrated and dried under vacuum to obtain the intermediate 5 (2.74 g crude product, 100%).

[¹H NMR (400 MHz, MeOD), absorption peaks in spectrum: δ 9.39 (s, 1H), 8.38 (s, 1H), 8.20-8.18 (dd, 1H), 7.83-7.79 (m, 1H), 7.42-7.37 (m, 2H), 7.10 (s, 1H), 5.38 (s, 2H), 3.97 (s, 3H); mass spectrum: m/s: [MH]⁺: 349.2]. The crude product of intermediate 5 was directly used in next step.

(2) preparation of intermediate 6: the solution of intermediate 2a (0.72 g, 3.9 mmol) in THF (10 mL) was slowly added to the intermediate 5 (0.5 g, 1.57 mmol) under stirring at 0° C.; then the mixture was slowly heated to room temperature and further stirred for 3 hours; subsequently saturated sodium bicarbonate was added to stop the reaction and dilute the mixture, and the resulting solution was extracted with ethyl acetate; finally the organic phase was washed with saturated sodium chloride solution, dried with anhydrous sodium sulfate, filtered, concentrated, dried under vacuum and purified by silica gel chromatograph to obtain intermediate 6 (450 mg, 61.6%) [m/s: [MH]⁺: 466.2].
(3) preparation of compound Ic: the intermediate 6 (450 mg, 0.97 mmol) was slowly added to the solution of N-methylbenzylamine (350 mg, 2.9 mmol) and potassium carbonate (268 mg, 1.94 mmol) in acetonitrile (20 mL); the mixture was stirred for 12 hours and then diluted with water, and extracted with ethyl acetate; the organic phase was washed with saturated sodium chloride solution, dried with anhydrous sodium sulfate, filtered, concentrated, dried under vacuum, and purified to get the title product (200 mg, 41%).

The obtained title product Ic was identified by ¹H-NMR (400 MHz, MeOD) and mass spectrum analysis, the results are as follows:

Absorption peaks in ¹H-NMR spectrum: δ 8.65 (s, 1H), 8.33 (s, 1H), 7.90-7.88 (dd, 1H), 7.56-7.54 (dd, 1H), 7.34-7.27 (m, 5H), 7.13-7.09 (t, 1H), 7.03-6.95 (m, 2H), 6.48-6.44 (d, 1H), 3.95 (s, 3H), 3.57 (s, 2H), 3.23-3.21 (d, 2H), 2.24 (s, 3H).

Wherein m/s: [MH]⁺: 506.2. Through calculation, it is confirmed that the product has the molecular formula of C₂₇H₂₅ClFN₅O₂, and the exact mass is 505.17.

### Embodiment 4

The compound of Formula Id has the following chemical formula:

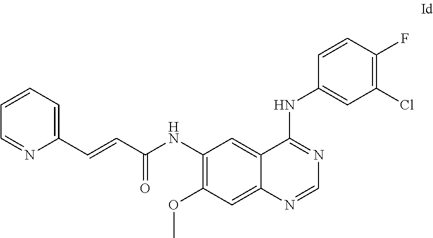 [SMILES: COc1cc2ncnc(Nc3ccc(F)c(Cl)c3)c2cc1NC(=O)/C=C/c1ccccn1]

This compound can be obtained by means of the substitution reaction of the intermediate 6 in the embodiment 3 with the intermediate 3a in the embodiment 2, and the specific preparation process can be seen in embodiment 3.

The obtained title product Id was identified by ¹H-NMR (400 MHz, MeOD) and mass spectrum analysis, the results are as follows:

Absorption peaks in ¹H-NMR spectrum: δ 10.04 (s, 1H), 9.90 (s, 1H), 9.08 (s, 1H), 8.73-8.72 (dd, 1H), 8.61 (s, 1H), 8.22-8.20 (dd, 1H), 7.97-7.93 (t, 1H), 7.89-7.86 (m, 1H), 7.75-7.68 (m, 3H), 7.51-7.46 (m, 2H), 7.37 (s, 1H), 4.10 (s, 3H).

Wherein m/s: [MH]⁺: 451.1. Through calculation, it is confirmed that the product has the molecular formula of C₂₃H₁₇ClFN₅O₂, and the exact mass is 449.11.

### Embodiment 5

The compound of Formula Ie has the following chemical formula:

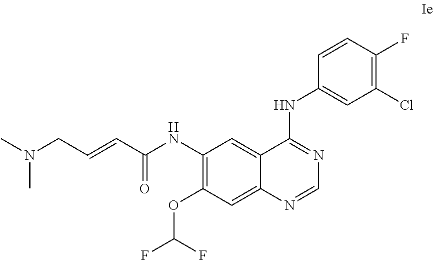 [SMILES: CN(C)C/C=C/C(=O)Nc1cc2c(Nc3ccc(F)c(Cl)c3)ncnc2cc1OC(F)F]

The compound of Formula Ie can be obtained using the following synthesis route:

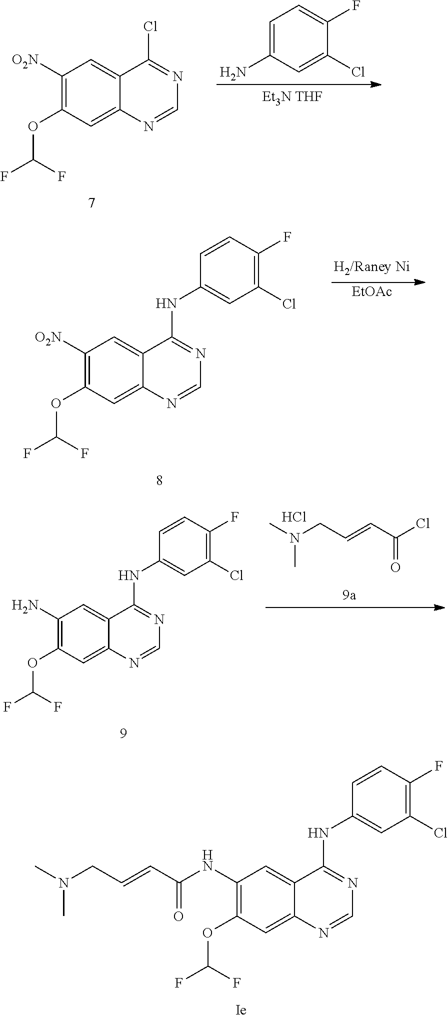

The preparation method of the compound of Formula Ie specifically comprises the steps of:

(1) preparation of intermediate 8: 3-chloro-4-fluoro-benzenamine (9.6 g, 66.1 mmol) was added into the intermediate 7 (18.1 g, 66.1 mmol) under stirring at room temperature; triethylamine (9.2 mL, 66.1 mmol) was added into the resulting solution, and the mixture was stirred at room temperature for 30 mins, diluted with water and extracted with ethyl acetate; finally the organic phase was washed with 1 N hydrochloric acid and saturated sodium chloride in sequence, dried with anhydrous sodium sulfate, filtered, concentrated, dried under vacuum, and purified by silica gel chromatograph to obtain the intermediate 8 (9 g, 35.4%).

[¹H NMR (400 MHz, DMSO), absorption peaks in spectrum: δ 10.39 (s, 1H), 9.43 (s, 1H), 8.81 (s, 1H), 8.17-8.15 (dd, 1H), 7.79-7.45 (t, j=68.8 Hz, 1H), 7.81 (s, 1H), 7.73 (s, 1H), 7.56-7.49 (s, 1H); mass spectrum, m/s: [MH]⁺: 385.7].

(2) preparation of intermediate 9: raney nickel (500 mg) was slowly added to the solution of intermediate 8 (5 g, 13.0 mmol) in ethyl acetate (100 mL) at room temperature; the mixture was stirred for 12 hours in the presence of H₂; subsequently the resulting solution was filtered, concentrated, dried under vacuum to get the intermediate 9 (4.5 g crude product, 97.8%) [m/s: MH]⁺: 355.2]. The intermediate 9 was directly used in next step.
(3) preparation of compound of Formula Ie: the solution of the intermediate 9a (3.9g, 21.2 mmol) in anhydrous tetrahydrofuran (10 mL) was slowly added to the intermediate 9 (4 g, 11.3 mmol); the mixture was slowly heated to room temperature and stirred for 3 hours; subsequently saturated sodium bicarbonate was added to stop the reaction and dilute the resulting mixture, the resulting solution was extracted with ethyl acetate; finally the organic phase was washed with saturated sodium chloride solution, dried with anhydrous sodium sulfate, filtered, concentrated, dried under vacuum, and purified by gel chromatograph to obtain the title product (2 g, 38%).

The obtained title product Ie was identified by H-nuclear magnetic resonance ¹H-NMR (400 MHz, MeOD) and mass spectrum analysis, the results are as follows:

Absorption peaks in ¹H-NMR spectrum: δ 8.91 (s, 1H), 8.51 (s, 1H), 8.03-8.01 (dd, 1H), 7.68-7.61 (dd, 1H), 7.50 (s, 1H), 7.33-6.97 (t, j=72 Hz, 1H), 7.26-7.22 (t, 1H), 7.06-6.99 (m, 1H), 6.52-6.49 (d, 1H), 3.30-3.21 (d, 2H), 3.32 (s, 6H).

Wherein m/s: [MH]^{±}: 466.2. Through calculation, it is confirmed that the product has the molecular formula of C₂₁H₁₉ClF₃N₅O₂, and the exact mass is 465.12.

Efficacy Test

### I. Enzyme Activity Inhibition Test of Compounds:

### 1. Test Method

Half maximal inhibitory concentration IC₅₀ (the concentration of a compound that is required for 50% inhibition of the activity of an enzyme) was tested based on an immobilized enzyme mixing with a specific substrate and the test compounds in different concentrations. The used test method was Caliper Mobility Shift Assay. The tested enzymes were EGFR and HER2, and the standard reference compounds were staurosporine. As a comparison, in the same test condition, the inhibitory effects of other similar compounds of Afatinib, Neratinib, and Gefitinib on EGFR kinase and HER2 kinase were tested.

According to the same test method, the inhibitory effects of the compound of Formula Ie on mutant EGFR kinases were tested, including single mutant EGFR^{LS58R}, EGFR^{T790M} and double mutant EGFR^{L858R/T790M} kinases.

### 2. Test Results

Table 1 shows the test results of inhibition of enzyme activity of compounds. The results show that the target compounds (Ia, Ib, Ic, Id and Ie) have very strong inhibitory effects on EGFR kinases. Meanwhile, the results show that the target compounds (Ia, Ib, Ic, Id and Ie) also have very strong inhibitory effects on HER2 kinases. Furthermore, the inhibitory effects of the compound of Formula Ie on the activity of EGFR kinases were higher than that of Afatinib, Neratinib and Gefitinib over two times.

TABLE 1
Test results of inhibition of enzyme activity of compounds

Inhibition of Kinase Activity (IC₅₀, nM)

| Compounds | EGFR | HER2 |
|---|---|---|
| Compound Ia | 2.0 | 13 |
| Compound Ib | 3.9 | 90 |
| Compound Ic | 1.3 | 15 |
| Compound Id | 5.1 | >10000 |
| Compound Ie | 0.40 | 11.7 |
| Afatinib | 1.0 | 10 |
| Neratinib | 1.2 | 1.3 |
| Gefitinib | 1.1 | 283 |
| Saurosporine | 93 | 304 |

Furthermore, by means of molecular biology methods, it is also demonstrated that the new EGFR inhibitor compounds can act on the interior of EGFR cells to competitively bind with ATP via covalent bonds, to achieve its irreversibility characteristics. The test results of reversible inhibition of enzyme activity of EGFR kinases show that the title compounds Ia and Ie has irreversibility.

Table 2 shows the test results of inhibition of compound of Formula Ie on mutant EGFR kinase activity. The results show that the compound of Formula Ie also has very strong inhibitory effects on three types of mutant EGFR kinases, including single mutant EGER^{L858R}, EGFR^{T790M} and double mutant EGFR^{L858R/1790M}.

TABLE 2
Test results of inhibitory effects of compound
Ie on three types of mutant EGFR kinases

Inhibition of Kinase Activity (IC₅₀, nM)

| Kinase Name | Compound Ie | Staurosporine |
|---|---|---|
| EGFR^{L858R} | 0.65 | 17 |
| EGFR^{T790M} | 4.6 | 0.86 |
| EGFR^{L858R/T790M} | 11 | 1.6 |

### II. Inhibition Test of Tumor Cells

### 1. Test Method

(1) Compounds: in vitro studies the test compounds was dissolved in 100% DMSO and diluted until the final concentration of DMSO was 0.1%. The 0.1% (v/v) DMSO was added into culture medium as solvent control. 9 concentration gradients were tested and each gradient was repeated twice.
(2) Tumor cell lines: the tumor cell lines were cultivated in RPMI10 culture medium containing 10% fetal calf serum in incubator at 37° C. in a 5% CO₂ air incubator. The tested tumor cell lines were: BT474, MDA-MB-231, SK-Br-3, A431, H292, H1975, HCC827, A549, H1650 and H1734.
(3) MTS method: cells were seeded into 96-well plate to give a density of about 3000 cells per well, and incubated overnight in a humidified incubator at 37° C. In the next day, the test compounds were added to the wells and further incubated for 72 hours. The cell activity was detected using MTS. IC₅₀ was calculated (the concentration of a compound that is required for 50% inhibition of the cell growth versus the DMSO control group, using nonlinear regression analysis of software GraphPad Prism).
(4) WST method: the cell activity was further demonstrated using more sensitive WST method. In a 96-well plate, each test compound was tested at 9 dosage concentrations and each dosage was made in triplicate. Samples were added in 100 μL/well. Cell control and negative control wells contained diluents instead of compounds. Except the negative control well, 100 μL cell suspension of complete medium was added into each test well, wherein corresponding cell numbers were included to assure that the cells in cell control well just cover the bottom of the well as the required incubation time was reached. The plate was incubated in a CO₂ incubator at 37° C. for 48 hours. The liquid in test wells was removed by pipetting. 100 μL CCK-8 (Cell counting Kit-8, purchased from DOJINDO Molecular Technologies) detection liquid (containing 10% CCK-8, 5% FBS corresponding medium) was added into each test well, then the plate was incubated in a CO₂ incubator under at 37° C. for 3 hours. OD value was detected by ELIASA at A450 nm. Cell viability percentage (%)=(O.D. test compound-O.D. blank)/(O.D. control-O.D. blank)×100. Mean value was calculated for each 3 repeated wells, and IC₅₀ was calculated using nonlinear regression (fitted curve) data analysis method.

Parts of test results of compound Ie listed in the table 3 and all of the test results listed in the table 4 were obtained by WST method.

### 2. Test Results

Inhibitory effects of title compound (Ia, Ib, Ic and Ie) on activity of tumor cells (BT474, MDA-MB-231, SK-Br-3, A431, BT474, H292, H1975, HCC827, MDA-MB-231, SK-Br-3, A549, H1650 and H1734) are shown in the table 3.

TABLE 3
Test results of inhibition of tumor cells

| Compounds | Tumor Cell Lines | IC₅₀ (μM) |
|---|---|---|
| Compound Ia | A431 | 4.914 |
| | BT474 | 0.264 |
| | H292 | 0.873 |
| | H1975 | 0.882 |
| | HCC827 | 0.015 |
| | MDA-MB-231 | ~76633 |
| | SK-Br-3 | 0.420 |
| | A549 | 5.932 |
| | H1650 | 6.569 |
| | H1734 | 1.343 |
| Compound Ib | A431 | 0.899 |
| | BT474 | 3.175 |
| | H292 | 0.502 |
| | H1975 | 30.79 |
| | HCC827 | 0.157 |
| | MDA-MB-231 | 4.013 |
| | SK-Br-3 | 3.099 |
| Compound Ic | A431 | 13.00 |
| | BT474 | 0.278 |
| | H292 | 0.727 |
| | H1975 | 0.715 |
| | HCC827 | 0.012 |
| | MDA-MB-231 | 13.89 |
| | SK-Br-3 | 0.418 |
| | A549 | >10 |
| | H1650 | >10 |
| | H1734 | 5.257 |
| Compound Id | BT474 | 2.138 |
| | H292 | 44.12 |
| | H1975 | >10000 |
| | HCC827 | 0.04 |
| | MDA-MB-231 | >10000 |
| | SK-Br-3 | 0.1553 |
| Compound Ie | A431 | 0.652 |
| | A431 (WST method) | 1.24 |
| | BT474 | 0.034 |
| | BT474 (WST method) | 0.088 |
| | H292 | 2.65 |
| | H292 (WST method) | 0.181 |
| | H1975 | 0.80 |
| | H1975 (WST method) | 0.198 |
| | HCC827 | 0.002 |
| | MDA-MD-231 | >10000 |
| | SK-Br-3 | 0.23 |
| | SK-Br-3 (WST method) | 0.028 |
| | A549 | 3.722 |
| | A549 (WST method) | 0.204 |
| | H1650 | 3.238 |
| | H1650 (WST method) | 0.702 |
| | H1734 | 0.539 |

It can be seen from the table 3 that, the compounds of the invention have inhibitory effect on activity of various tumor cells, wherein H1975, A549, H1650 and H1734 were drug-resistant tumor cell lines for Gefitinib and Erlotinib.

Further tests demonstrate that compound Ie also has strong inhibitory effects on other various indications of tumor cells, parts of the test results are listed in the table 4.

TABLE 4
test results of inhibition effects of compound
Ie on various indications of tumor cells

| Sources of Tumor Tissue | Tumor Cell Lines | IC₅₀(μM) |
|---|---|---|
| Lung | NCI-H358 | 0.195 |
| | NCI-H2126 | 1.685 |
| | Calu-3 | 0.022 |
| Breast | MDA-MB-468 | 0.076 |
| | ZR-75-30 | 0.683 |
| Colon | CW-2 | 0.036 |
| Uterine Neck | C-33A | 0.057 |
| Stomach | NUGC-3 | 0.055 |
| | NCI-N87 | 0.001 |
| Prostate | DU145 | 0.853 |
| Head and Neck | FaDu | 0.169 |
| | CAL27 | 0.002 |
| Kidney | ACHN | 0.107 |

### III. Tests of Inhibitory Effects of Transplanted Tumors in Nude Mice

### 1. Test Method

H1975 human non-small cell lung cancer tumor cells were inoculated into 18 nude mice (BALB/c, male, 5-6 weeks of age), and when the mean tumor volume reaches 150 mm³ the nude mice were randomized into three groups, including: control group (5 nude mice), 20 mg compound Ie/kg/day dose group (8 nude mice) and 75 mg Gefitinib/kg/day (5 nude mice). Test compounds were continuously administrated orally for 21 days. Tumor size and body weight of nude mice were recorded twice weekly from the first day of administration. Tumor weight was calculated using the formula (1×w²), wherein 1 and w respectively represents the maximum and minimum size in each measurement. The change relation of mean tumor volume with the days after tumor transplantation was graphed according to the calculation results.

Furthermore, more pharmacological models of tumor cell lines in nude mice were utilized to validate the inhibitory effects of compound Ie. The test method was specifically described as follows: H1975, A549, HCC827, A431, N87, MDA-MD-468 and CAL27 tumor cells respectively were inoculated into nude mice (BALB/c, male, 5-6 weeks of age), and when the mean tumor volume reaches 150 mm³ mice was randomized into groups. The H1975 tumor model in nude mice includes one positive control group and three dosage groups of compound Ie (6.7 mg/kg/day, 13.4 mg/kg/day, and 20.1 mg/kg/day. The A549 tumor model in nude mice includes one positive control group, two dosage groups of compound Ie (10 mg/kg/day, 20 mg/kg/day), and one dosage group of Docetaxel (12 mg/kg/day). The HCC827 tumor model in nude mice includes one positive control group, two dosage groups of compound Ie (0.5 mg/kg/day, 2.5 mg/kg/day), and one dosage group of Erlotinib (12.5 mg/kg/day). The A431 tumor model in nude mice includes one positive control group, two dosage groups of compound Ie (2.5 mg/kg/day, 5 mg/kg/day), and one dosage group of Erlotinib (50 mg/kg/day). The N87 tumor model in nude mice includes one positive control group, two dosage groups of compound Ie (5 mg/kg/day, 10 mg/kg/day), and one dosage group of Lapatinib (90 mg/kg/day, administration twice daily). The MDA-MD-468 tumor model in nude mice includes one positive control group, two dosage groups of compound Ie (5 mg/kg/day, 10 mg/kg/day), and one dosage group of Docetaxel (12 mg/kg/week) and one dosage group of Afatinib (30 mg/kg/day). The CAL27 tumor model in nude mice includes one positive control group and one dosage group of compound Ie (10 mg/kg/day). Compounds were continuously administrated orally for 21 days (wherein Docetaxel was injected intravenously once weekly). The tumor size and body weight of nude mice were recorded twice weekly from the first day of administration. The tumor volume was calculated using formula (1×w²), wherein 1 and w respectively represents the maximum and minimum size in each measurement. The change relation of the mean tumor volume with days after tumor transplantation was graphed according to the calculation results.

### 2. Results

The initial test results show (table 2) that: the compound Ie of the invention has good therapeutic effect on H1975 tumor model of non-small cell lung cancer, and inhibits the growth of H1975 cells (T/C % is 59%). Gefitinib has no therapeutic effect on H1975 tumor model of nude mice, and the growth speed of H1975 cells was similar to the control group.

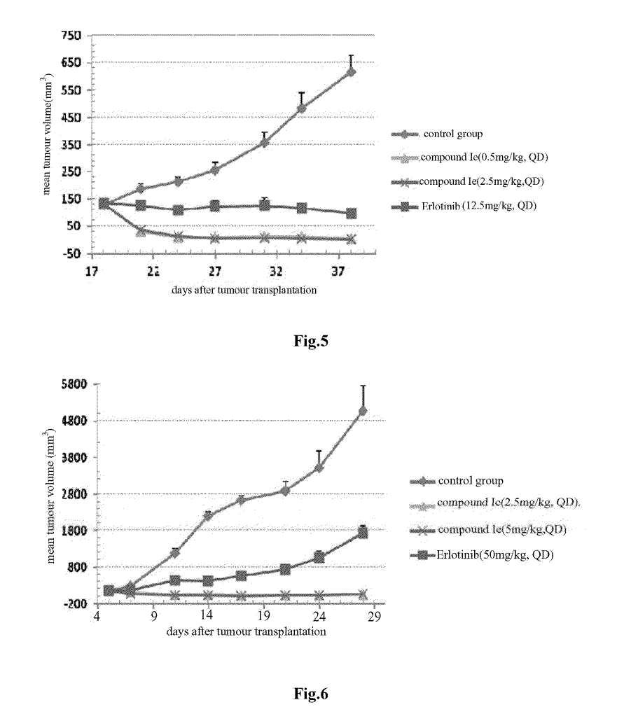

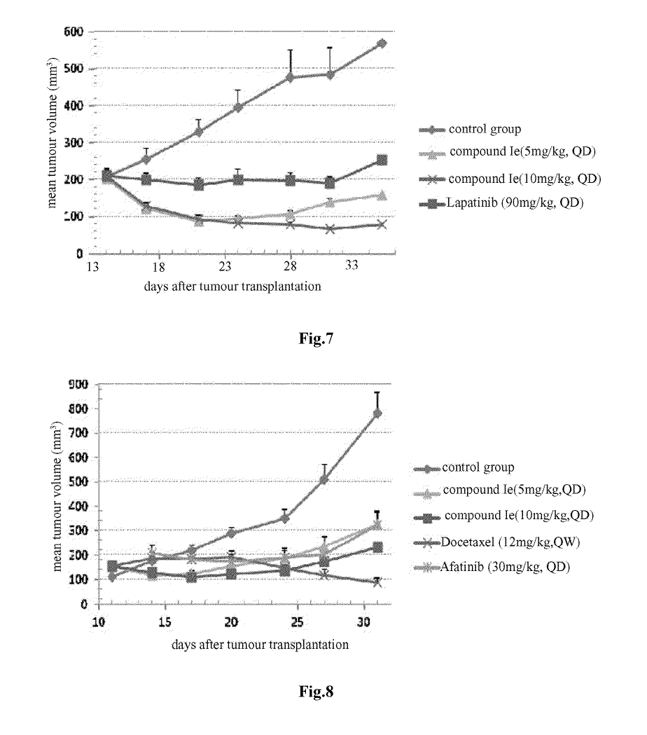

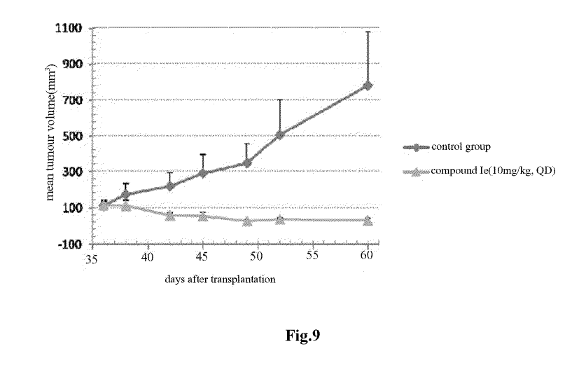

Further verification test results of pharmacological model in nude mice were shown in FIGS. 3-9. The test of human tumor cells transplanted tumor in nude mice shows that the compound Ie of the invention has strong inhibitory effects and therapeutic effects on tumor models of H1975 (non-small cell lung cancer), A549 (non-small cell lung cancer), HCC827 (non-small cell lung cancer), A431(EGFR), N87(gastric cancer), MDA-MD-468 (breast cancer) and CAL27 (head and neck cancer) in a lower dosage. Compound of Formula Ie has stronger inhibitory effects on tumor cell growth than other similar targeted drugs (such as Erlotinib, Lapatinib and Afatinib) or the cytotoxic chemotherapeutic agent Docetaxel in tumor models such as HCC827, A431, N87, MDA-MD-468 and A549.

### IV Pharmacokinetic Experiments

### 1. Experiment Method

Experiment animals: CD-1 mice, male, 6-7 weeks of age; body weight: 20-25 g; Preparation of samples: the test compounds respectively were formulated to 0.2 mg/mL (for intravenous administration) and 1.0 mg/mL (for oral administration) for use. Administration routes: oral administration/intravenous administration. Amount and frequency of administration: 5 mL/kg for single administration.

Sample collection: blood was collected at the time points of 5 min, 15 min, 30 min, 1 h, 2 h, 4 h, 8 h and 24 h, about 0.5-1.0 mL whole blood was collected from 3 animals at each time point. All of the animals were euthanized after the experiments.

### 2. Sample Analysis and Results

Sample analysis: the collected samples were detected by LC-MS/MS, and the instrument model is SHIMADZU20A-API4000.

Pharmacokinetic data analysis: the obtained plasma concentration data were fitted and calculated using WinNolin according to non-compartment model. Partial results are shown in table 5.

TABLE 5
pharmacokinetic parameters of title compounds
calculated according to non-compartment model

| | | Pharmacokinetic | | | |
|---|---|---|---|---|---|
| Dosage | Administration | Parameters | Compound | Compound | Compound |
| (mg/kg) | Route | (unit) | Ia | Ic | Ie |
| 1 | Intravenous | CL (L/hr/kg) | 9.18 | 5.23 | 6.90 |
| | Injection | Vss (L/kg) | 5.47 | 14.7 | 18.9 |
| | N = 3 | Terminal t_{1/2} (hr) | 0.729 | 2.63 | 2.31 |
| | | AUCₗₐₛₜ(hr*ng/mL) | 104 | 163 | 137 |
| | | AUC_{INF}(hr*ng/mL) | 110 | 206 | 167 |
| | | MRT_{INF}(hr) | 0.616 | 3.01 | 2.88 |
| 5 | Oral | Tmax (hr) | 1.17 | 2.00 | 4.33 |
| | Administration | Cmax (ng/mL) | 31.4 | 66.7 | 92.3 |
| | N = 3 | Terminal t_{1/2} (hr) | 1.76 | N/A | 7.11 |
| | | AUCₗₐₛₜ | 91.4 | 283 | 1150 |
| | | (hr*ng/mL) | | | |
| | | AUC_{INF}(hr*ng/mL) | 140 | N/A | 1600 |

The experiment results in CD-1 mice show that the compounds of the invention have good pharmacokinetic characteristics, including properties such as low clearance, high oral absorption, long half-life and high tissue distribution. The compounds of the invention have excellent physical and chemical properties and good druggability.

### V Long-Term Toxicity Experiment (28 Days)

### 1 Experiment Method

Experiment Animals: rats, male, 6-8 weeks of age, body weight: 180-250 g; dogs, male, about 24 months, body weight: 7-10 kg.

Preparation of sample: in rat's experiments the mixed formulation powder of the compound Ie was formulated into 0.1, 0.3 and 1 mg/mL homogeneous suspension for use; administration route: oral administration; amount and frequency of administration: 10 mL/kg, once daily for 28 days. In dog's experiments the tablets of compound Ie were fed to dogs. Afatinib was formulated to 1 or 2 mg/kg homogeneous suspension to feed to rats or dogs.

Sample collection: blood was collected at the time points of 1, 10, 20 and 28 days respectively, about 120-200 μL whole blood was collected from 3 animals at each time point. Blood was collected at 1 h, 4 h, 8 h and 24 h after administration.

### 2. Sample Analysis and Results

Sample analysis: the collected samples were detected by LC-MS/MS, and the instrument model was SHIMADZU20 A-API4000.

Pharmacokinetic data analysis: the obtained plasma concentration data were fitted and calculated using WinNolin according to non-compartment model.

### 3. Experiment Results

In rats, AUC (area under the concentration versus time curve) of the compound Ie is about 5 times higher than that of Afatinib in the same dosage, AUC of 1 mg/kg compound Ie is equivalent to AUC of 5 mg/kg Afatinib. In rat's experiments, no toxicity reaction was observed in animals of 1 mg/kg compound Ie group, and there was significant toxicity reaction in animals of 5 mg/kg Afatinib group under the same AUC. Furthermore, no toxicity reaction was observed in animals of 3 mg/kg compound Ie group, and there was very significant toxicity reaction in animals of 10 mg/kg Afatinib group under similar AUC.

In dog's experiments, in the same dosage AUC (area under the concentration versus time curve) of the compound Ie was similar to Afatinib. No toxicity reaction was observed in animals of 1 mg/kg compound Ie group, and there were toxicity reactions in animals of both 5 mg/kg compound Ie group and 5 mg/kg Afatinib group, and the toxicity reaction of animals of Afatinib group was greater than the compound Ie group.

The toxicity reaction of compound Ie is reversible, and the animals can recover after drug withdrawal. In rat's experiments, the animals of compound Ie groups (1 mg/kg, 3 mg/kg and 10 mg/kg) regain weight very quickly, and all of the animals have weight close to control group at 5-6 days after drug withdrawal. Afatinib group recovers more slowly, and particularly, the animals of high dosage group of 10 mg/kg Afatinib have reduced weight during the recovery phase after drug withdrawal.

Generally, in the long-term (28 days) toxicity experiments of rats and dogs with daily administration, in the same dosage, the compound of Formula Ie has higher safety and lower toxicity than other similar compounds (such as Afatinib).

The above embodiments are described only as representative example. It can be seen from the above embodiments that, the compounds of the invention are ideal high effective, dual and irreversible tyrosine kinase inhibitors, and it is expected that they can treat or prevent various malignancy diseases, such as breast cancer, ovarian cancer, gastrointestinal cancer, oesophageal cancer, lung cancer, head and neck squamous cancer, pancreatic cancer, epidermis squamous cancer, prostatic cancer, neuroglioma and nasopharynx cancer, and achieve excellent effects, and even they can be combined with different pharmaceutical salts to form oral formulations (such as tablets or capsules). The tablets or capsules prepared from the compounds of the invention can be taken one or more times daily. The compounds of the invention also can be combined with other drugs to form compound formulations.

The above embodiments are described for illustrating the technical concept and features of invention, the aim is intended to enable a person skilled in the art to appreciate the content of the invention and further implement it, and the protecting scope of the invention can not be limited hereby. Also, any equivalent variations or modifications made according to the spirit of the invention should be covered within the protecting scope of the invention.

1. A compound of the Formula (I), a pharmaceutically acceptable salt or hydrate thereof, a prodrug thereof, or a metabolite thereof produced in any type of metabolism, 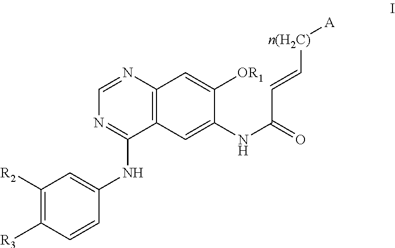
wherein:
R₁ is —CH₂F, —CHF₂, C₂-C₁₂ fluorinated hydrocarbyl, C₂-C₁₂ chlorinated hydrocarbyl; or
R₁ is —CₖH₂ₖ₋₁O, wherein k is an integer between 3 and 6;
R₂ is F, Cl, C₁-C₁₂ saturated alkyl or C₂-C₁₂ unsaturated alkenyl or alkynyl or alkenynyl;
R₃ is F, Cl, —OCᵢH₂ᵢC₅H₄N, —OCᵢH₂ᵢ C₄H₃N₂ or —OCᵢH₂ᵢC₃H₂N₃, wherein i is an integer between 1 and 4;
n is 0, 1, 2 or 3;
A is a 5-membered or 6-membered, saturated or unsaturated, substituted or unsubstituted heterocyclic structure, or a substituted or unsubstituted benzene ring; or
A is —NR₅R₆, wherein R₅ and R₆ are independently selected from H and C₁-C₁₂ hydrocarbyl; and
in the compound of the Formula (I), the pharmaceutically acceptable salt or hydrate thereof, the prodrug thereof, or the metabolite thereof produced in any type of metabolism, non-exchangeable hydrogen is not substituted, or partly or fully substituted by deuterium.

2. The compound of the Formula (I), the pharmaceutically acceptable saltor hydrate thereof, the prodrug thereof, or the metabolite thereof produced in any type of metabolism as claimed in claim 1, wherein in the Formula (I), R₁ is selected from the group consisting of —CHF₂, chlorinated ethyl and tetrahydrofuryl, and wherein R₂, R₃, n and A are defined as in claim 1.

3. The compound of the Formula (I), the pharmaceutically acceptable salt or hydrate thereof, the prodrug thereof, or the metabolite thereof produced in any type of metabolism as claimed in claim 2, wherein R₁ is —CHF₂ or

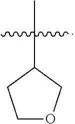 [SMILES: *C1CCOC1 ; "."]

4. The compound of the Formula (I), the pharmaceutically acceptable salt or hydrate thereof, the prodrug thereof, or the metabolite thereof produced in any type of metabolism as claimed in claim 1, wherein in the Formula (I), R₂ is Cl, R₃ is F; and wherein R₁, n and A are defined as in claim 1.

5. The compound of the Formula (I), the pharmaceutically acceptable salt or hydrate thereof, the prodrug thereof, or the metabolite thereof produced in any type of metabolism as claimed in claim 1, wherein in the Formula (I), n is 0 or 1; and wherein R₁, R₂, R₃ and A are defined as in claim 1.

6. The compound of the Formula (I), the pharmaceutically acceptable salt or hydrate thereof, the prodrug thereof, or the metabolite thereof produced in any type of metabolism as claimed in claim 1, wherein in the Formula (I), A is selected from the group consisting of —N(CH₃)₂, —N(CH₃)CH₂CH₃, —N(CH₃)CH₂Ph, imidazolyl, pyridyl, unsubstituted and alkyl-substituted butyrolactam and valerolactam, and wherein R₁, R₂, R₃ and n are defined as in claim 1.

7. The compound of the Formula (I), the pharmaceutically acceptable salt or hydrate thereof, the prodrug thereof, or the metabolite thereof produced in any type of metabolism as claimed in claim 6, wherein A is

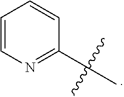 [SMILES: *c1ccccn1 ; "."]

8. The compound of the Formula (I), the pharmaceutically acceptable salt or hydrate thereof, the prodrug thereof, or the metabolite thereof produced in any type of metabolism as claimed in claim 1, wherein the compound is selected from the compounds respectively having the following formulas:

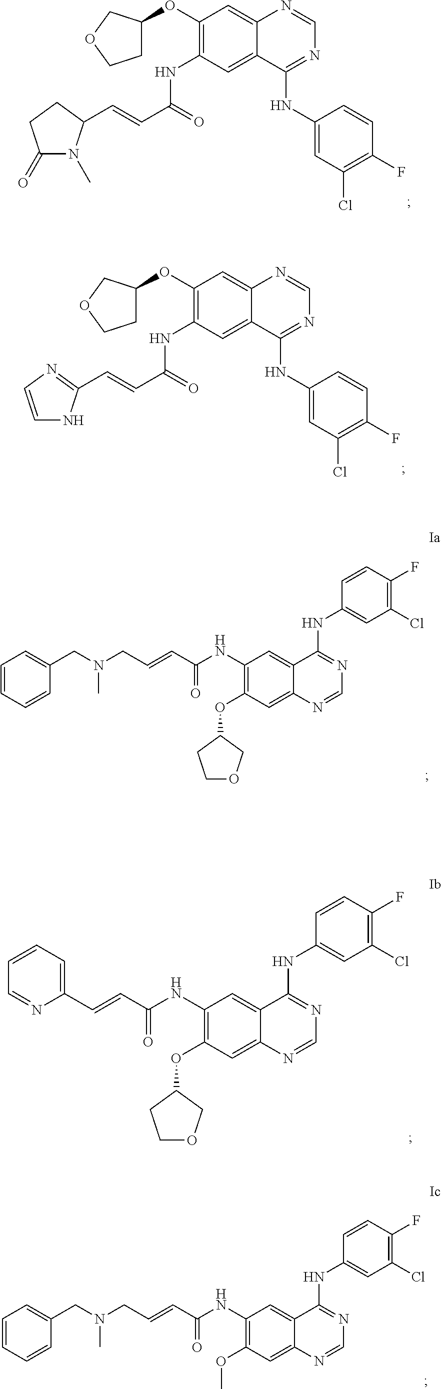

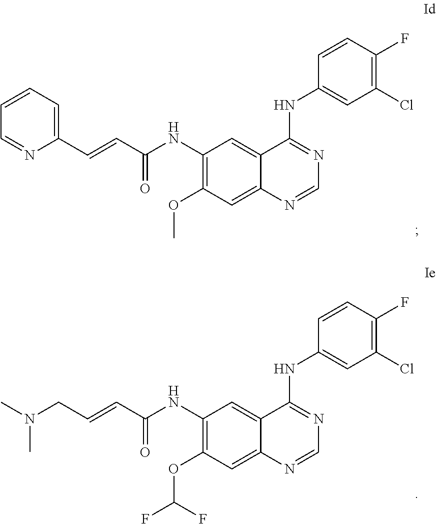 [SMILES: COc1cc2ncnc(Nc3ccc(F)c(Cl)c3)c2cc1NC(=O)/C=C/c1ccccn1 ; CN(C)C/C=C/C(=O)Nc1cc2c(Nc3ccc(F)c(Cl)c3)ncnc2cc1OC(F)F]

9. (canceled)

10. (canceled)

11. A method of preventing at least one indication associated with EGFR and HER2 kinase functions in a subject in need thereof, comprising administering an effective amount of the Formula (I), the pharmaceutically acceptable salt or hydrate thereof, the prodrug thereof, or the metabolite thereof produced in any type of metabolism as claimed in claim 1.

12. The method of claim 11, wherein the indication associated with EGFR and HER2 kinase functions is breast cancer, ovarian cancer, gastrointestinal cancer, oesophageal cancer, lung cancer, head and neck squamous cancer, pancreatic cancer, epidermis squamous cancer, prostatic cancer, neuroglioma, or nasopharynx cancer.

13. A method of treating at least one indication associated with EGFR and HER2 kinase functions in a subject in need thereof, comprising administering an effective amount of a the Formula (I), the pharmaceutically acceptable salt or hydrate thereof, the prodrug thereof, or the metabolite thereof produced in any type of metabolism as claimed in claim 1.

14. The method of claim 13, wherein the indication associated with EGFR and HER2 kinase functions is breast cancer, ovarian cancer, gastrointestinal cancer, oesophageal cancer, lung cancer, head and neck squamous cancer, pancreatic cancer, epidermis squamous cancer, prostatic cancer, neuroglioma, or nasopharynx cancer.
